(19) United States
(12) Patent Application Publication
(10) Pub. No.: US 2024/0327053 A1
(43) Pub. Date: Oct. 3, 2024

(54) RECEPTACLE

(71) Applicant: Multigate Medical Products Pty Ltd, Villawood (AU)

(72) Inventors: Abdu MOUKAHAL, Villawood (AU); Amy LEEDHAM, Villawood (AU); Caitlin NEIL, Villawood (AU); Bob SMIT, Villawood (AU)

(21) Appl. No.: 18/579,464

(22) PCT Filed: Jul. 14, 2022
(86) PCT No.: PCT/AU2022/050737

§ 371 Date: Jan. 15, 2024

(30) Foreign Application Priority Data
Jul. 15, 2021 (AU) 2021902173

(51) Int. Cl. B65D 1/26; B65D 21/02; B65D 25/56; G01F 22/00

(52) U.S. Cl. CPC B65D 1/26; B65D 21/0233; B65D 25/56; G01F 22/00

(57) ABSTRACT

A receptacle (10) is provided having a base portion (12) and at least one wall (16) extending from and surrounding the base portion (12). The at least one wall (16) terminates in a rim (14). The at least one wall (16) is formed with stepped gradations (18) between the base portion (12) and the rim (14).

### TECHNICAL FIELD

The present invention relates to receptacles suitable for use in medical environments.

### BACKGROUND OF THE INVENTION

The following references to and descriptions of prior proposals or products are not intended to be and are not to be construed as, statements or admissions of common general knowledge in the art. In particular, the following prior art discussion does not relate to what is commonly or well known by the person skilled in the art, but assists in the understanding of the inventive step of the present invention of which the identification of pertinent prior art proposals is but one part.

In medical environments, receptacles, such as dishes, trays and bowls, are used for a wide variety of purposes. While there is a need for receptacles to be sterile and fit for purpose, cost demands tend to make mass manufactured single use plastic materials a popular choice. However, environmental concerns cause consumers to require receptacles which reduces the environmental impact of the use of single use plastic.

The present invention seeks to provide receptacles which are suitable for purpose in a medical environment and permit manufacturing which reduces environmental impact compared with single use plastic materials and which may ameliorate the foregoing shortcomings and disadvantages or which will at least provide a useful alternative.

### SUMMARY OF THE INVENTION

According to one aspect of the invention, there is provided a receptacle 10 having a base portion 12 and at least one wall 16 extending from and surrounding said base portion 12, said at least one wall 16 terminating in a rim 14, wherein said at least one wall 16 is formed with stepped gradations 18 between said base portion 12 and said rim 14.

Preferably, an abutment surface 20 is formed extending from a surface of said at least one wall 16 below said rim 14, wherein when said receptacle 10 is stacked with another receptacle 10 the rim 14 of one receptacle 10 abuts the abutment surface 20 thereby providing a clearance between the walls 16 of the stacked receptacles 10 which prevents the stacked receptacles 10 from being wedged together.

In exemplary embodiments the receptacle 10 is thermoformed from recycled polypropylene.

### BRIEF DESCRIPTION OF THE DRAWINGS

The invention may be better understood from the following non-limiting description of a preferred embodiment, in which:

FIG. 1 is an illustration of a range of receptacles 10 of preferred embodiments of the present invention;

FIGS. 2A & 2B is an illustration of a pair of receptacles 10 before and after stacking according to one embodiment;

FIG. 3 shows a cross-section of the stacked receptacles 10 of FIG. 2B clearly showing the abutment arrangement 20; and

FIG. 4 shows a further cross-section of the stacked receptacles 10 of FIG. 2B.

### DETAILED DESCRIPTION OF PREFERRED EMBODIMENT

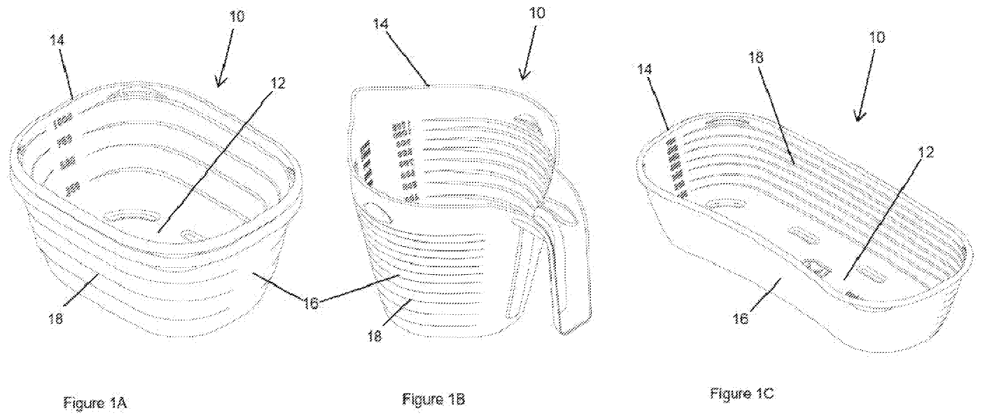

As shown in FIGS. 1A-1C, receptacles 10 of different shapes and sizes can be provided for embodiments of the present invention. Different shapes and sizes suit different medical purposes.

Common to each embodiment is a base portion having side walls 16 extending from and surrounding the base portion 12 and terminating with an upper rim 14. The upper rim 14 is preferably formed as a curved lip to facilitate ease of gripping and carrying the receptacle 10. Receptacles having, for example, a rectilinear shape such as the receptacle shown in FIG. 1A, may have a wider lip formed on the shorter sides to promote ease of gripping at the wider lip portion.

As shown, the walls 16 of the receptacle are formed with stepped gradations 18. This overall formation permits the use of thermoforming processes in the manufacture of the receptacle 10. The stepped gradations 18 provide strength and rigidity for the thermoformed structure. The receptacle 10 may be formed from recycled plastic material, such as polypropylene, thereby reducing environmental impacts from mass manufacturing. However, it will be appreciated that alternative suitable materials and manufacturing processes can be employed.

The positions of the stepped gradations 18 can be selected to conveniently coincide with particular fluid volumes of contents of the receptacle as visual measurement indicator. For example, the 1 litre receptacle of FIG. 1A is shown having stepped gradations 18 at the 250 ml, 500 ml, 750 ml and 1 litre levels.

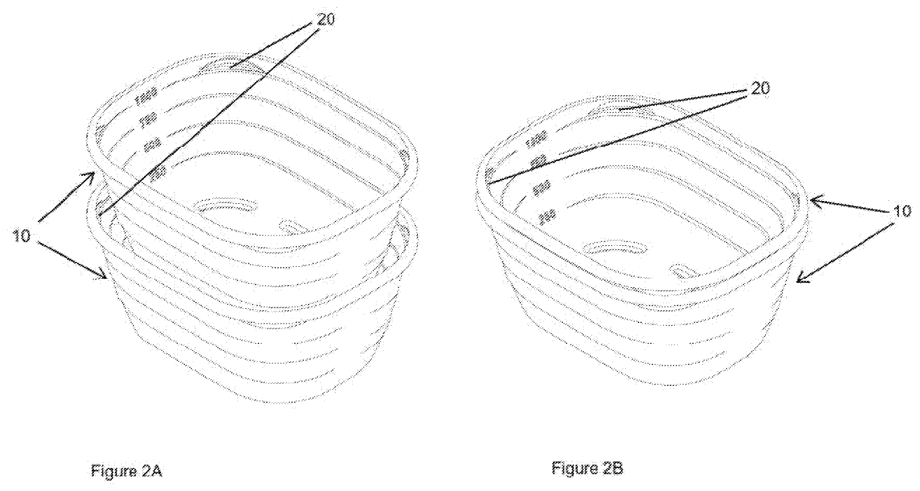

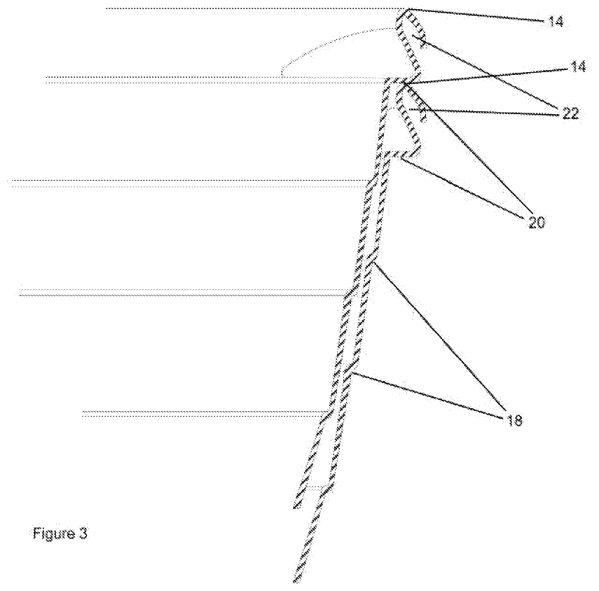

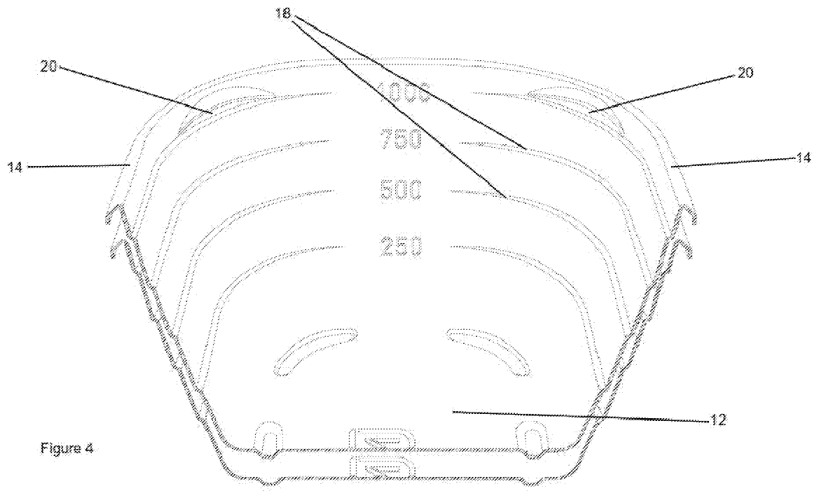

As shown in FIGS. 2-4, the receptacles 10 are intended to be able to be stacked for ease of storage. However, in medical environments, for example, it is important that a receptacle 10 can be retrieved from a stack without difficulty. Consequently, it is preferred that the stacked receptacles are not able to wedge with each other. To account for this, an abutment surface 20 is formed extending from a surface of the wall 16 below the rim 14. When receptacles 10 are stacked, the rim 14 of one receptacle 10 abuts the abutment surface 20 of the other receptacle. As shown, this arrangement provides a clearance between the walls 16 of the stacked receptacles 10 and prevents the receptacles 10 becoming wedged together. The abutment surface 20 may conveniently be formed as part of an upper stepped gradation. As further shown, the arrangement of the abutment surface 20 also provides an indent 22 within the curved lip of the rim 14 and above the abutment surface 20. This indent 22 further facilitates an easy gripping portion for a person carrying the receptacle. As shown, preferably a plurality of abutment surfaces 20 are provided on each receptacle 10. In the case of the rectilinear shaped receptacle in FIG. 1A, each corner of the receptacle 10 is provided with an abutment surface 20.

The term “comprise” and variants of that term such as “comprises” or “comprising” are used herein to denote the inclusion of a stated integer or integers but not to exclude any other integer or integers, unless in the context or usage an exclusive interpretation of the term is required.

Those skilled in the art will appreciate that the invention described herein is susceptible to variations and modifications other than those specifically described. All such variations and modifications are to be considered within the scope and spirit of the present invention the nature of which is to be determined from the foregoing description.

1. A receptacle having a base portion and at least one wall extending from and surrounding said base portion, said at least one wall terminating in a rim, wherein said at least one wall is formed with stepped gradations between said base portion and said rim.

2. The receptacle of claim 1, wherein said stepped gradations are selectively positioned to provide fluid volume indicators for contents of said receptacle.

3. The receptacle of claim 1, wherein an abutment surface is formed extending from a surface of said at least one wall below said rim, wherein when said receptacle is stacked with another receptacle the rim of one receptacle abuts the abutment surface thereby providing a clearance between the walls of the stacked receptacles which prevents the stacked receptacles from being wedged together.

4. The receptacle of claim 3, wherein said abutment surface is formed as part of an upper one of said stepped gradations.

5. The receptacle of claim 3, wherein said receptacle is provided with a plurality of abutment surfaces.

6. The receptacle of claim 5, wherein each of said plurality of abutment surfaces is arranged at a respective corner part of said receptacle.

7. The receptacle of claim 1, wherein said receptacle is formed of recycled plastic material.

8. The receptacle of claim 1, wherein said receptacle is formed of polypropylene material.

9. The receptacle of claim 1, wherein said receptacle is manufactured by thermoforming.
